(19) United States
(12) Patent Application Publication
(10) Pub. No.: US 2014/0349861 A1
(43) Pub. Date: Nov. 27, 2014

(54) Method for Distinguishing Biological Material Products

(71) Applicant: Sunpower Technologies LLC, San Marcos, CA (US)

(72) Inventor: Travis Jennings, San Diego, CA (US)

(73) Assignee: Sunpower Technologies LLC, San Marcos, CA (US)

(21) Appl. No.: 13/900,073

(22) Filed: May 22, 2013

(51) Int. Cl. C12Q 1/68

(52) U.S. Cl. CPC C12Q 1/6895
USPC 506/7; 435/6.12; 427/427.7; 427/384; 427/2.1; 156/185

(57) ABSTRACT

A method for encoding and identifying biological materials is disclosed. The method may include encoding and identifying plants from which controlled substances may be derived and other materials for which movement and distribution may need to be tracked. The biological material may be first encoded using DNA oligomers. A spray method or the use of an encoded substrate, both using these DNA oligomers for encoding the biological material, may be employed. The biological material, or a part of the biological material, may be first encoded by atomizing a solution containing DNA oligomers onto it and then dried by an appropriate method. Thereafter, the part of the encoded biological material, or the nitrocellulose substrate, may be dissolved with a buffer solution for extracting the DNA oligomers. Then, the dissolved solution may be used for generating a barcode by a suitable detection scheme.

### BACKGROUND

1. Field of the Disclosure

The present disclosure relates generally to biological encoding systems, and more particularly to DNA barcodes for distinguishing, tracking, and controlling biological material such as certain plants and seeds.

2. Background Information

Many agricultural products may need to be regulated for being authenticated, verified, tracked, and controlled to prevent the cultivation, manufacturing, distribution, and sale of unauthorized biological material products that are considered illegal. Some of these biological material products, from which controlled substances are derived, may include plants such as cannabis plants, coca plant, opium poppy, khat, and iboga, among others. Another, biological material product that may need to be regulated may include genetically-modified seeds which are protected by legally enforced plant-breeders and other intellectual property rights.

A variety of plants and seeds may be confused with those plants and seeds that are used for medical roles in human society, or genetically-modified seeds that are authorized for being sold or resold by farmers. For example, according to the variety of cannabinoid content (active ingredients) and other compounds of cannabis plants, these can be classified as toxic or non-toxic for human consumption. Several cannabis plants with non-toxic cannabinoids can be used as a physician-recommended form of medicine or herbal therapy; however, some cannabis plants that may include toxic cannabinoids may cause negative effects, such as problems with memory and learning. These toxic cannabinoids may be considered illegal.

There are few methods for identifying legal biological material products from illegal varieties; however, there are certain methods that may modify these biological material products or their production, which may be considered neither convenient nor accurate, and may represent a high cost for several regulation entities. For example, the use of genetic engineering may innately modify the plant in a very fundamental form.

There is therefore a need to be able to distinguish authorized biological material products from common, illegal toxic varieties of biological material products; a new method may be applied to perform the identification of legal and illegal biological materials with more accuracy and lower cost.

### SUMMARY

According to various embodiments of the present disclosure, a method for encoding and identifying biological materials, such as plants and seeds, may be disclosed. This method may allow to encode and identify plants from which controlled substances, such as cocaine, heroin, and marijuana, may be derived. Furthermore, this method may be applied to encode and identify biological material for which movement and distribution may need to be controlled and tracked, such as genetically-modified seeds.

In this disclosure, two different methods may be described for encoding biological materials. One method to encode biological materials may include using a spray method. The spray method may be performed by a dispensing device with a reservoir of a barcoded solution. This barcoded solution may include DNA oligomers combined with a suitable solution such as TE buffer (Tris EDTA pH 8). The barcoded solution may be in charge of encoding the biological material to be utilized in a later analysis.

Another method to encode biological materials includes the use of an encoded substrate such as nitrocellulose. The nitrocellulose substrate may include DNA oligomers and may be wrapped around biological material when packaging.

The samples obtained by the encoding methods may be soaked and dissolved in a buffer solution to extract the encoded DNA oligomers. This dissolved solution may be utilized for identifying the type of biological material by employing a suitable detection scheme.

The type of encoded DNA oligomers detected by common detection schemes may be compared against a database to translate the meaning of the encoded DNA oligomer sequences. Additional information about the biological material may be obtained after having the detection results, including but not limited to plant breed, growth facility, lot number, and expiration date, among others.

This method for distinguishing legal and illegal biological material may allow perform an accurate analysis and detection without altering biological material properties.

In one embodiment, a method for encoding a biological material comprises forming a barcode solution including a sequence of DNA oligomers, wherein the sequence of DNA oligomers encodes information about the biological material; and atomizing the barcode solution on the biological material using a dispensing device.

In another embodiment, a method for encoding a biological material comprises forming an encoded substrate including a nitrocellulose substrate encoded with a sequence of DNA oligomers, wherein the sequence of DNA oligomers encodes information about the biological material; drying at least a part of the biological material; and adhering the dried part of the biological material to the encoded substrate.

In yet another embodiment, a method for encoding a biological material comprises forming an encoded substrate including a nitrocellulose substrate encoded with a sequence of DNA oligomers, wherein the sequence of DNA oligomers encodes information about the biological material; and wrapping the encoded substrate around a section of the biological material.

In another embodiment, a method for distinguishing biological material comprises encoding biological material with a sequence of DNA oligomers, wherein the sequence of DNA oligomers forms encoded information about the biological material; dissolving encoded samples of the biological material in a buffer solution to extract the DNA oligomers from the encoded samples; detecting the DNA oligomers using a detection scheme to form a readout describing the sequence of DNA oligomers; and comparing the sequence of DNA oligomers to a database to translate the meaning of the sequence of DNA oligomers, wherein the DNA sequence describes encoded information about the biological material.

Additional features and advantages of an embodiment will be set forth in the description which follows, and in part will be apparent from the description. The objectives and other advantages of the invention will be realized and attained by the structure particularly pointed out in the exemplary embodiments in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIGS. 1A to 1C describe methods for encoding biological material using DNA oligomers. FIG. 1A describes a spray method for encoding biological materials using DNA oligomers, according to an embodiment. FIG. 1B describes an encoded substrate for encoding biological material with DNA oligomers, according to an embodiment. FIG. 1C shows an embodiment of an encoded substrate using a suitably-sized strip.

FIG. 2 describes a method for extracting and detecting encoded DNA oligomers using samples obtained in FIGS. 1A to 1C, according to an embodiment.

FIG. 3 illustrates an encoding process that a pharmaceutical laboratory may follow to encode a stem of a medical cannabis plant, according to an exemplary embodiment.

FIG. 4 illustrates an encoding process that an agricultural biotechnology corporation may follow to encode seeds that are genetically modified, according to an exemplary embodiment.

FIG. 5 illustrates an encoding process that a pharmaceutical laboratory may follow to encode a leaf of a coca plant for medical purposes, according to an exemplary embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### Definitions

As used here the following terms may have the following definitions:

“DNA oligomer” refers to a short single-stranded sequence of deoxyribonucleic acid (DNA) formed by bounded molecules.

“Coding strand” refers to a synthetic short single-stranded sequence of DNA used to encode cannabis plants.

“Barcode” refers to a pattern that allows the identification or verification of the type of a living being based on a DNA sequence.

“Biological material” refers to substances containing genetic information from organisms of the Plantae kingdom, such as plants and seeds, capable of reproducing themselves or being reproduced in a biological system.

### DESCRIPTION OF THE DRAWINGS

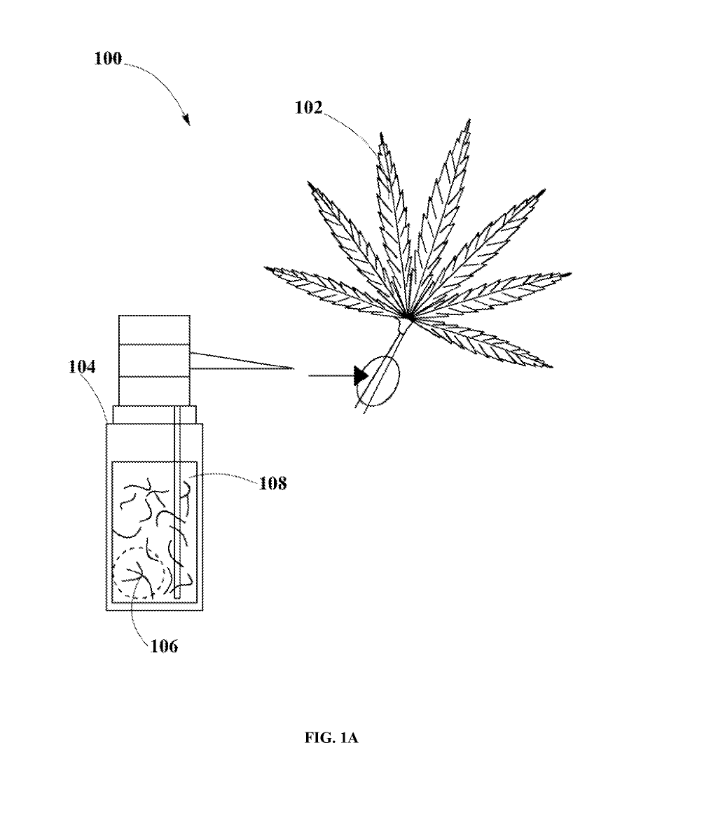

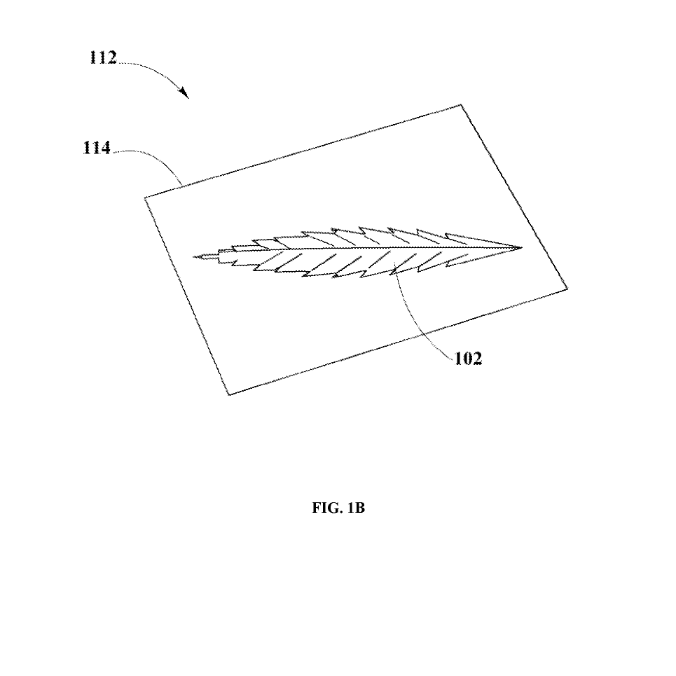

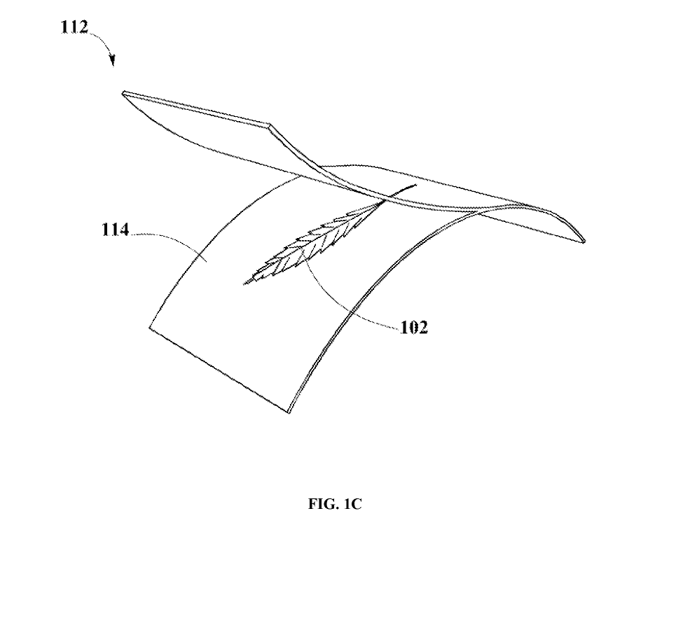

FIGS. 1A to 1C describe methods for encoding biological material using synthetic DNA oligomers. According to an embodiment, these encoding methods with DNA oligomers may allow to identify plants and seeds that are legal from illegal varieties. FIG. 1A describes an encoding method using a spray solution with DNA oligomers; and FIG. 1B describes an alternative encoding method using an encoded substrate with oligomers. However, these two methods may not intend to limit the disclosure, other methods may be applied to encode biological materials.

The sequences of DNA oligomers used in FIG. 1A and FIG. 1B may be agreed upon by a standards committee. This standards committee may have an agreement and cooperation among different parties of interest such as law enforcement, distributors, manufacturers, pharmacies, end users, and others entities.

Furthermore, the diverse types of DNA oligomers which may be used to encode each biological material may be according to specific information such as breed, lot number, growth facility, expiration date, and among others.

FIG. 1A describes a spray method 100 for encoding biological materials. In this embodiment, a plant 102, such as a cannabis plant, may be encoded with synthetic DNA oligomers; nevertheless, this spray method 100 may also be used for other biological materials.

In order to encode plants 102 using spray method 100, particular types of DNA oligomers may be needed, as well as, a suitable solution, and a dispensing device 104.

The coding strands (CS 106) of DNA oligomers, which may be used to encode plant 102, may preferably be between about 20 to about 50 base pairs in length. Each strand of each DNA oligomer may be at a concentration of at least about 1 μM to about 50 μM. Further, a minimum of about 100 picomols of each CS 106 may be deposited onto a detectable area of plant 102, where this amount may be approximately from about 50 μL of 2 μM solution.

The suitable solution utilized in spray method 100 may be appropriate for solubilizing DNA oligomers and avoiding problems such as degradation. This solution may be TE buffer (Tris EDTA pH 8) which must be freshly autoclaved. Alternatively, distilled water (dH2O) may be used. In the suitable solution, one or more DNA oligomers with characteristics described above may be included and deposited into a dispensing device 104. The mixture of the suitable solution with DNA oligomers may produce a barcoded solution 108.

The dispensing device 104, which may be employed in spray method 100 for depositing the barcoded solution 108 to plant 102, may be capable of reproducibly depositing controllable quantities of the CS 106 from the barcoded solution 108.

The spray method 100 may be employed when a detectable part of plant 102 may be atomized with barcoded solution 108 using dispensing device 104. Subsequently, the suitable atomized part of plant 102 may require to be dried for a long-term storage. This drying process may be performed by applying different methods such as exposing plant 102 to air in for a determined amount of time or using a desiccator device. The determined concentration covered with barcoded solution 108 may be used as a sample for later analysis.

FIG. 1B describes an encoded substrate 112 with DNA oligomers for encoding biological materials. A suitable substrate such as nitrocellulose substrate 114, which may be encoded by CS 106 of DNA oligomers, may be used in this embodiment. Additionally, in this embodiment encoded substrate 112 may encode plant 102; however, this encoded substrate 112 may also be used for other biological materials.

This encoding process may begin when a part of plant 102 may be first dried by different methods mentioned in FIG. 1A. Subsequently, the dried part of plant 102 may be adhered into a surface of nitrocellulose substrate 114 encoded with DNA oligomers.

Alternatively, in FIG. 1C, a suitably-sized strip of nitrocellulose substrate 114 may be encoded with DNA oligomers. A suitable section of plant 102 may be adhered to or wrapped around the encoded suitably-sized strip at any point either during or after manufacture, prior to shipping plant 102 to a customer.

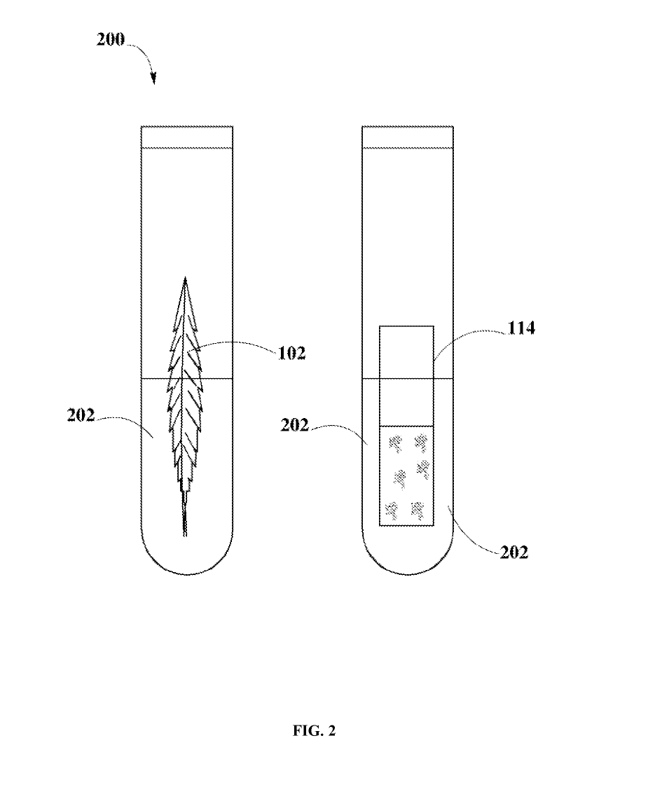

FIG. 2 describes a DNA oligomer extraction and detection method 200 using samples obtained in FIG. 1.

After encoding biological materials by methods mentioned in FIG. 1, the corresponding encoded samples may be analyzed by detecting and validating the encoded DNA oligomers of these biological materials.

The encoded samples, such as the spray-encoded section of the plants 102 obtained in FIG. 1A, and nitrocellulose substrate 114 used in FIG. 1B, may be soaked and dissolved in an appropriate buffer solution 202. This buffer solution 202 may be phosphate buffered saline (PBS), where the volume to be used may vary from about 0.1 mL to about 5 mL for an appropriate amount of time from about 30 seconds to about 3 minutes. Buffer solution 202 may extract the encoded DNA oligomers from the encoded samples of biological materials, which may be used to analyze these products.

Subsequently, the dissolved solution obtained by the mixture of buffer solution 202 and DNA oligomers may be optionally filtered through a common 0.22 μm syringe filter. The syringe filter may remove unnecessary particles that may affect the detection of DNA oligomers during a decoding method. In another embodiment, a filter integrated into an assay device may be used to detect the type of biological material.

Thereafter, the encoded DNA oligomers may be detected by common detection schemes, such as lateral flow assays, microarray detection, polymerase chain reaction (PCR), and solution-based Förster Resonance Energy Transfer (FRET) assays, among others. If all of the appropriately encoded DNA oligomers are present and detectable on the sample, then readout from the chosen detection system may be compared to a database to translate the meaning of the DNA oligomer sequences detected.

The presence or absence of particular encoded DNA oligomer sequences may allow entities to distinguish, track, and control the biological material. When DNA oligomers are decoded, certain information about the biological material may be obtained, including plant breed, growth facility, lot number, and expiration date, among others.

### EXAMPLES

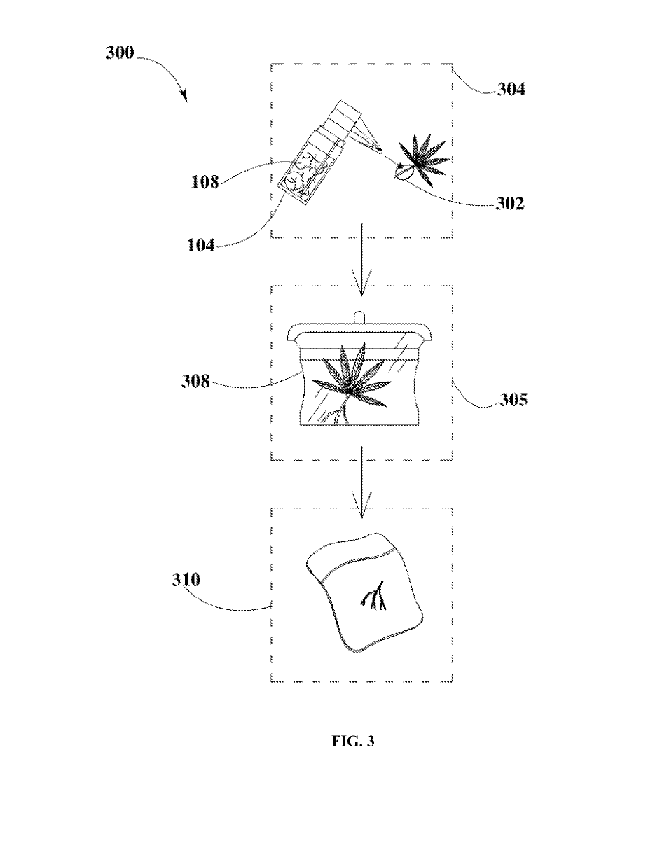

In Example #1, FIG. 3 describes an encoding process 300 that a pharmaceutical laboratory may follow to encode a stem 302 of a medical cannabis plant. The encoding process 300 of stem 302 may be applied before packaging and selling it to a customer for accomplishing the regulatory medicinal controls. Stem 302 is sprayed 304 with about 50 μL to about 100 μL of a concentrated barcoded solution 108 containing oligomers that corresponds to a breed type, lot number, and expiration dates of cannabis plant, from where stem 302 was extracted. The barcoded solution 108 is deposited on the surface of stem 302 using a dispensing device 104 having a spray nozzle module. Then, the spray-encoded stem 302 may be dried 305 using a desiccator device 308. Finally, stem 302 may be ready to be packaged 310 and sold to the customer.

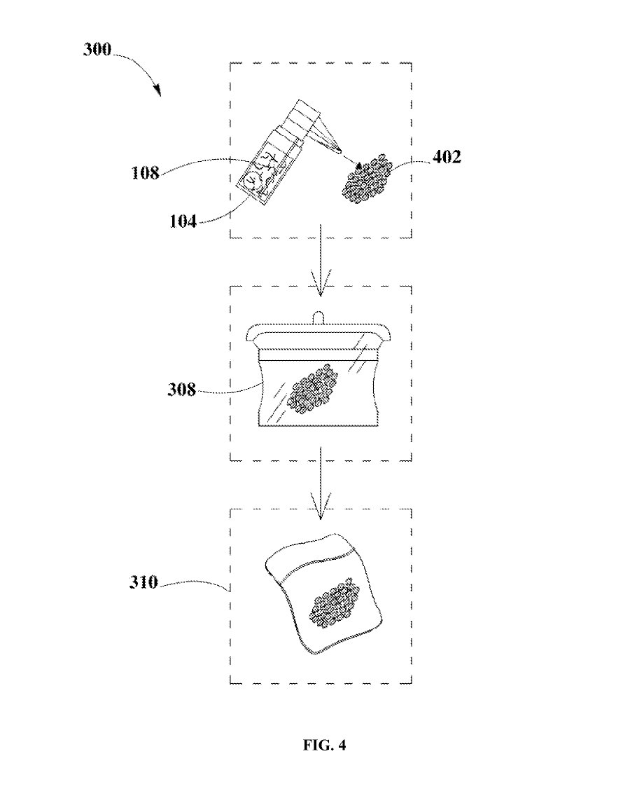

In Example #1, FIG. 4 describes an encoding process 300 that an agricultural biotechnology corporation may follow to encode seeds 402 that are genetically modified. The encoding process 300 of seeds 402 may be applied before packaging and selling them to a customer for accomplishing the regulatory controls. Seeds 402 are sprayed 304 with about 50 μL to about 100 μL of a concentrated barcoded solution 108 containing oligomers that corresponds to a breed type, lot number, and expiration dates of seeds 402. The barcoded solution 108 is deposited on the surface of seeds 402 using a dispensing device 104 having a spray nozzle module. Then, the spray-encoded seeds 402 may be dried 305 using a desiccator device 308. Finally, seeds 402 may be ready to be packaged 310 and sold to the customer.

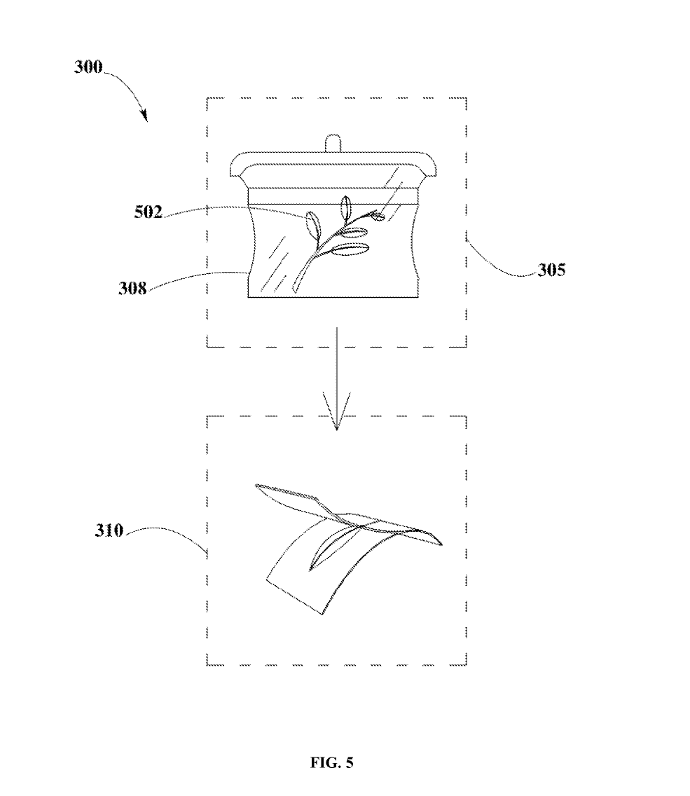

In Example #1, FIG. 5 describes an encoding process 300 that a pharmaceutical laboratory may follow to encode a leaf 502 of a coca plant for medical purposes. The encoding process 300 of leaf 502 may be applied before packaging and selling it to a customer for accomplishing the regulatory medicinal controls. Leaf 502 may be dried 305 using a desiccator device 308. Finally, leaf 502 may be ready to be packaged 310 and sold to the customer.

While various aspects and embodiments have been disclosed, other aspects and embodiments are contemplated. The various aspects and embodiments disclosed are for purposes of illustration and are not intended to be limiting, with the true scope and spirit being indicated by the following claims.

The embodiments described above are intended to be exemplary. One skilled in the art recognizes that numerous alternative components and embodiments that may be substituted for the particular examples described herein and still fall within the scope of the invention.

What is claimed is:

1. A method for encoding a biological material comprising:
forming a barcode solution including a sequence of DNA oligomers, wherein the sequence of DNA oligomers encodes information about the biological material; and
atomizing the barcode solution on the biological material using a dispensing device.

2. The method of claim 1, wherein atomizing the biological material comprises exposing only a portion of the biological material to the barcode solution.

3. The method of claim 2, further comprising:
drying the portion of the biological material by exposing the portion of the biological material to air for a predetermined amount of time.

4. The method of claim 2, wherein the portion is a stem of the biological material.

5. The method of claim 1, wherein the sequence of DNA oligomers is between 20 to 50 base pairs in length.

6. The method of claim 1, wherein each DNA oligomer is at a concentration of at least 1 μM to 50 μM.

7. The method of claim 1, wherein the solution is TE buffer (Tris EDTA pH 8).

8. The method of claim 1, wherein the solution is distilled water.

9. The method of claim 1, wherein the encoded information describes the biological material's breed, lot number, growth facility, composition of medicinal ingredients, dosage of medicinal ingredients, date of manufacture, or expiration date.

10. A method for encoding a biological material comprising:
forming an encoded substrate including a nitrocellulose substrate encoded with a sequence of DNA oligomers, wherein the sequence of DNA oligomers encodes information about the biological material;
drying at least a part of the biological material; and
combining the dried part of the biological material to the encoded substrate.

11. The method of claim 10, wherein the encoded information describes the biological material's breed, lot number, growth facility, or expiration date.

12. The method of claim 10, wherein the sequence of DNA oligomers is between 20 to 50 base pairs in length.

13. The method of claim 11, wherein each DNA oligomer is at a concentration of at least 1 μM to 50 μM.

14. A method for encoding a biological material comprising:
forming an encoded substrate including a nitrocellulose substrate encoded with a sequence of DNA oligomers, wherein the sequence of DNA oligomers encodes information about the biological material; and
wrapping the encoded substrate around a section of the biological material.

15. The method of claim 14, wherein the encoded information describes the biological material's breed, lot number, growth facility, composition of medicinal ingredients, dosage of medicinal ingredients, date of manufacture, or expiration date

16. The method of claim 14, wherein the sequence of DNA oligomers is between 20 to 50 base pairs in length.

17. The method of claim 14, wherein each DNA oligomer is at a concentration of at least 1 μM to 50 μM.

18. A method for distinguishing biological material comprising:
encoding biological material with a sequence of DNA oligomers, wherein the sequence of DNA oligomers forms encoded information about the biological material;
dissolving encoded samples of the biological material in a buffer solution to extract the DNA oligomers from the encoded samples;
detecting the DNA oligomers using a detection scheme to form a readout describing the sequence of DNA oligomers; and
comparing the sequence of DNA oligomers to a database to translate the meaning of the sequence of DNA oligomers, wherein the DNA sequence describes encoded information about the biological material.

19. The method of claim 18, wherein the detection scheme is lateral flow assays, microarray detection, polymerase chain reaction, or solution-based Forester Resonance Energy Transfer (FRET) assays.

20. The method of claim 18, wherein the encoded information describes the biological material's breed, lot number, growth facility, or expiration date.

21. The method of claim 18, wherein the buffer solution is phosphate buffered saline (PBS).

22. The method of claim 21, wherein the volume of the PBS is from 0.1 mL to 5 mL.

23. The method of claim 18, wherein the encoded samples soak and dissolve in the buffer solution for 30 seconds to 3 minutes.

24. The method of claim 18, further comprising:
filtering the buffer solution including the encoded samples through a 0.22 μm syringe filter to remove unnecessary particles.
